(19) United States
(12) Patent Application Publication
(10) Pub. No.: US 2019/0336722 A1
(43) Pub. Date: Nov. 7, 2019

(54) Systems And Methods For Enhanced Therapy Optimization In Connection With Multisensory Wellness In Light And Sound

(71) Applicant: LYT Events, Inc., Plainsboro, NJ (US)

(72) Inventors: Douglas Donaldson, Plainsboro, NJ (US); J. Scott Omelianuk, Hoboken, NJ (US); Chris Obssuth, Paramus, NJ (US)

(73) Assignee: LYT Events, Inc., Plainsboro, NJ (US)

(21) Appl. No.: 16/402,860

(22) Filed: May 3, 2019

Related U.S. Application Data
(60) Provisional application No. 62/666,327, filed on May 3, 2018.

(51) Int. Cl. A61M 21/02; A61N 5/06

(52) U.S. Cl. CPC A61M 21/02; A61M 2021/0027; A61N 2005/0626; A61N 5/0618

(57) ABSTRACT

A system for enhanced therapy optimization in connection with multisensory wellness in light and sound comprising a database storing a library of sounds and a processor in communication with the database. The system generates a sound healing methodology and a light healing methodology based on a determined condition of a patient, and provides the patient with a therapy program of light and sound by synching a generated sound scape and a generated light scape. The system receives, in response to the provided therapy program, feedback from the patient in real time and changes the provided therapy program based on the received feedback from the patient in real time.

### CROSS-REFERENCE TO RELATED APPLICATIONS

This application claims priority to U.S. Provisional Patent Application No. 62/666,327, filed on May 3, 2018, the entire disclosure of which is hereby expressly incorporated by reference.

### BACKGROUND

### Technical Field

The present disclosure relates generally to the field of therapy using light and sound. More particularly, the present disclosure relates to systems and methods for enhanced therapy optimization in connection with multisensory wellness in light and sound.

### Related Art

There currently is a public mental health crisis in the United States and in many countries around the world. There are various ways in treating mental health such as prescribing medications including antidepressants and other drugs. For a number of reasons, it might be desirable to provide an alternate means of therapy to an individual with a mental health condition. One potential therapeutic solution is to subject a patient to certain light and sound patterns to heal the patient.

Current prior art solutions for providing light and sound therapy to a patient have many drawbacks. For example, these prior art solutions do not tailor the light and sound therapy to the particular patient's personal mental condition, prevailing cultural interests and evidence-based guidelines. Accordingly, these current solutions provide a single light and sound therapy program that are not effective to a patient because it does not address the patient's specific unique characteristics. Furthermore, the current solutions do not take into account real time feedback from patients to provide enhanced therapeutic benefits to the patients.

Therefore, in view of existing technology in this field, what would be desirable is an enhanced system and method for providing light and sound therapy that can be tailored to a patient and can change in real time based on how the patient is responding to the therapy.

### SUMMARY

A method is provided which includes the step of determining or receiving a condition of a patient to be treated, prevailing cultural interests, and evidence-based guidelines. The method further includes the step of generating a sound healing methodology and a light healing methodology based on the condition of the patient, prevailing cultural interests, and evidence-based guidelines. The method also includes the step of generating a sound scape from a library of sounds by selecting an instrument, a frequency, an interval, and a volume of the sound scape based on the sound healing methodology. The method additionally includes the step of generating a light scape from a plurality of light characteristics of color, brightness, saturation, shape, duration, or movement. Still further, the method includes the step of generating a therapy program of light and sound to be provided to a patient by synching the light scape with the sound scape to generate the therapy program. Moreover, the method includes the step of receiving a request to provide the therapy program to the patient. As will be explained in greater detail below, this can include the ability to change the length of the sound and/or light program. Also, the method includes the step of providing the therapy program to a delivery device for providing light and sound therapy to the patient. The method includes the step of receiving feedback of the patient's vital signs or input feedback in real time. Finally, the method includes the step of changing the therapy program in real time based on the patient's vital signs or input feedback.

### BRIEF DESCRIPTION OF THE DRAWINGS

The foregoing features of the invention will be apparent from the following Detailed Description, taken in connection with the accompanying drawings, in which:

FIG. 1 is a diagram illustrating a process for generating an audio and light program for providing therapy to a patient;

FIG. 2 is a diagram illustrating a process for optimizing the audio and light programs for providing therapy to the patient;

FIG. 3 is a diagram illustrating an audio library for generating an audio program;

FIG. 4 is a diagram illustrating a light augmentation process for generating a light program;

FIG. 5 is an example graphical user interface for selecting details of a sound and light therapy program provided by the systems and methods of the present disclosure;

FIG. 6 is a drawing illustrating a healing bowl for providing light and sound therapy to a patient;

FIG. 7 is diagram illustrating hardware and software components of a system of the present disclosure; and

FIG. 8 is a diagram illustrating hardware and software components of a computer system on which the system of the present disclosure could be implemented.

### DETAILED DESCRIPTION

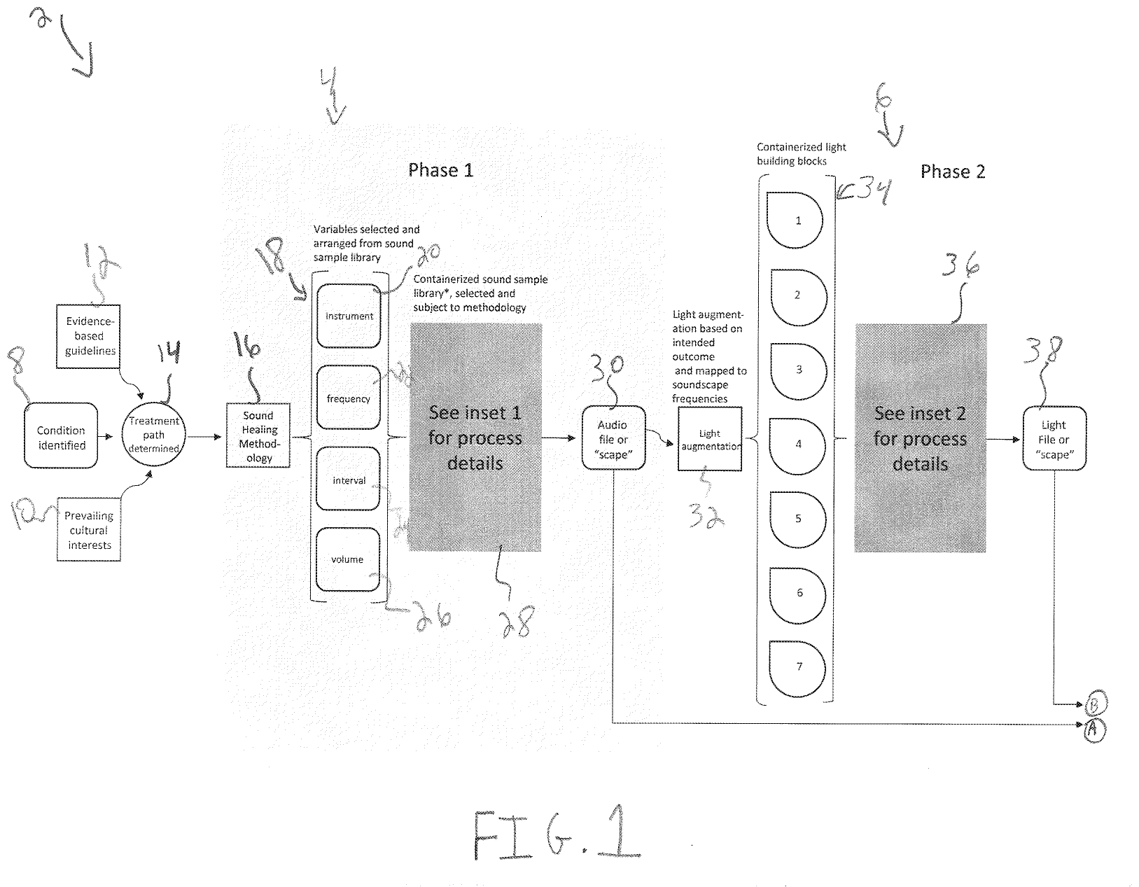

The present disclosure relates to systems and methods for enhanced therapy optimization in connection with multisensory wellness in light and sound, as discussed in detail below in connection with FIGS. 1-8.

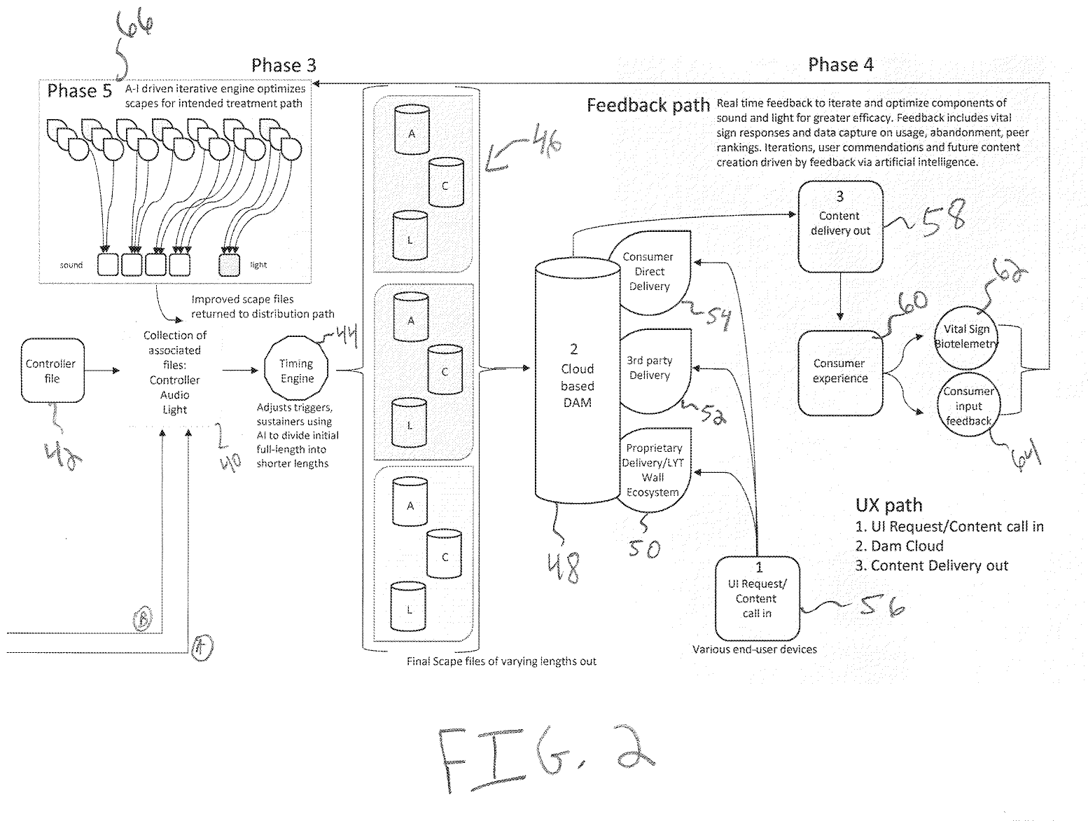

The present disclosure provides a system and method for generating light and sound programs for providing therapy to a patient. Example embodiments of the systems and methods of the present disclosure include five phases which will be explained greater detail below. Two example phases will be explained with principal reference to FIG. 1 and three example phases will be explained with principal reference to FIG. 2.

The present disclosure discloses providing therapy to a patient. However, the present disclosure is not limited to providing therapy to a patient. Rather, the present disclosure can include “users” who are not in need of medical treatment. These users can use the systems and methods of the present disclosure for other purposes such as to reach a more creative state or to relax. Moreover, a group of users can use the systems and methods of the present disclosure to synch the group of users towards some motivation. This group exercise can occur at a sales meeting, for example.

FIG. 1 is a diagram illustrating an example process 2 for generating an audio and light program for providing therapy to a patient. The process 2 includes an example first phase 4 for generating the audio program and an example second phase 6 for generating the light program. The systems and methods of the present disclosure can provide only sound or only light. The systems and methods of the present disclosure are not limited to providing both sound and light programs to a user. The first phase 4 can take as input a condition 8 of a patient to receive the treatment, prevailing cultural interests 10 which can be continually updated over time, and evidence-based guidelines 12 which can also be continually updated over time. Alternatively, the patient can select his or her condition 8 and the system can receive this selection as input. These components can be used to generate a treatment path 14 to provide therapy to the patient. The prevailing cultural interests 10 can include information relating to which sounds are popular, types of popular guided meditation, popular colors, and other similar types of information. The evidence-based guidelines 12 can include research information relating to which types of sound or lights can be used to treat a certain health problem.

The first phase 4 can include a sound healing methodology 16 which can be generated based on the treatment path 14. The treatment path can include the specifics of the meditation sound and light to be used on a patient (e.g., one sustained sound note or a plurality of different notes). Based on the patient's particular condition 8, the prevailing cultural interests 10, and evidence-based guidelines 12, the sound healing methodology 16 can be generated which is specifically and uniquely tailored to each individual patient. The sound healing methodology 16 can be used to select a plurality of variables 18. The plurality of variables 18 can include, but is not limited to, an instrument 20, a frequency 22, an interval 24, and volume 26. The plurality of variables 18 can be used as input to an audio library 28 for generating an audio file 30 or “audio scape” which can be played to the patient for therapy. The audio library 28 can include real sounds or the system can electronically generate sounds. The audio library 28 can include base tones and frequencies to human voices and traditional healing instruments (e.g., Himalayan Bowls, Native American Flute, Shruti Box, etc.). Each sound sample can be tagged with appropriate metadata and can be thematically categorized, allowing for easy retrieval of each element to meet the needs of each soundscape. Sounds can be specifically chosen based on characteristics that will deliver the appropriate positive emotional outcome.

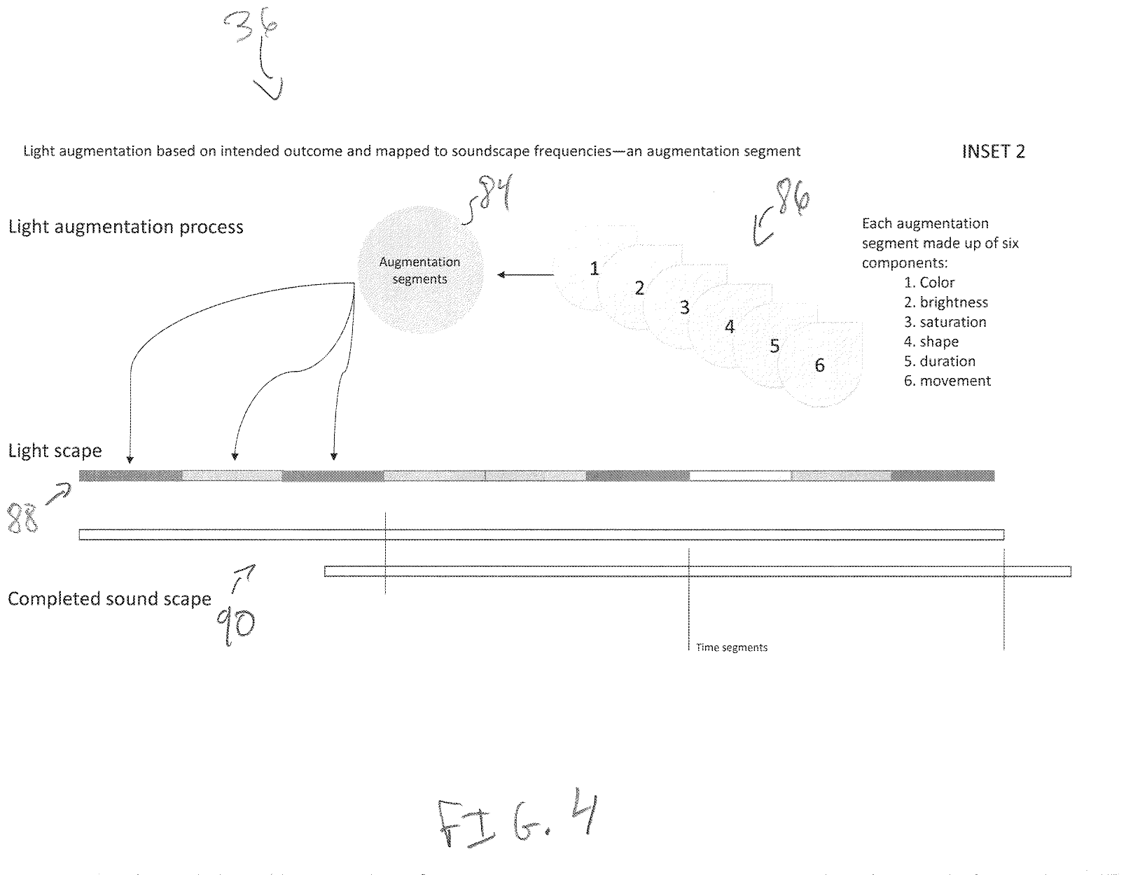

The second phase 6 for generating a light program for therapy will now explained in greater detail. The second phase 6 includes a light augmentation step 32 which takes as input the audio file 30 generated in the first phase 4 and/or the treatment path 14. By using the treatment path 14, the second phase 6 can generate a light program that is specifically and uniquely tailored to each individual patient because the treatment path 14 takes into account the patient's particular condition 8, the prevailing cultural interests 10, and evidence-based guidelines 12. Moreover, the light augmentation step 32 can generate a light program that is tailored to and coincides with the audio file 30 to provide the desired therapeutic effects. The light augmentation step 32 can use a plurality of light building blocks 34. The plurality of light building blocks 34 can include a number of different characteristics of light. These characteristics can include, but are not limited to, color, brightness, saturation, shape, duration, and movement. Other characteristics can be used in the systems and methods of the present disclosure. The second phase 6 can then use a light augmentation process 36 which can generate a light file 38 or “light scape” for being displayed to the patient. The light augmentation process 36 can take as input the plurality of light building blocks 34 and the light augmentation step 32 to generate the light file 38. The light augmentation process 36 will be explained in greater detail below in connection with FIG. 4. The systems and methods of the present disclosure can include a light library with light and color samples and moving images from a RGB palette hues and also moving objects (e.g., circles or other geometric shapes to natural elements of rain clouds, etc.). Each light sample can be appropriately tagged with metadata and can be thematically categorized, allowing for easy retrieval of each element to meet the needs of each light scape. Light and color elements can be specifically chosen to complement the audio tracks based on characteristics that will deliver the appropriate positive emotional outcome.

FIG. 2 is a diagram illustrating a process for optimizing the audio and light programs for providing therapy to the patient. As noted above, FIG. 2 illustrates example phase 3, example phase 4, and example phase 5 of the example process for an enhanced system and method of providing light and sound therapy to a patient. In phase 3, the audio file 30 and the light file 38 can be collected in a collection of associated files 40 in which a controller file 42 can synch the audio file 30 and the light file 38 and can include metadata for performing such functions. The synch function allows the audio and light to match so that the user experiences the intended therapy as light and sound working together in a certain combination can be important to achieve certain therapeutic effects. The collection of associated files 40 can be received by a timing engine 44 where the files are adjusted using artificial intelligence to divide the initial full-length files into a plurality of files 46 sorted by audio, light and controller, where these files can be of varying shorter lengths of audio and sound than the collection of associated files 40. The plurality of files 46 can be stored in a cloud-based data asset management database 48. As noted above, each completed long-form sound and light scape can be processed and placed in a traditional wave-form timeline. From the original file, the systems and methods of the present disclosure can mix and match finished containers of sound/light to create additional shorter pieces that maintain the integrity and efficiency of the original work.

In example phase 4, delivery of the light and sound is provided to a patient in need of light and sound therapy. The systems and methods of the present disclosure can include a plurality of light and sound therapy delivery means including, but not limited to, proprietary delivery systems 50, third party delivery systems 52, and consumer direct delivery systems 54. Proprietary delivery systems 50 can include a number of different delivery systems such as a wall for producing light or a healing bowl for producing light and sound as will be explained in greater detail below. The wall can include lights to provide the light program and can also include speakers installed in the wall to provide sound with the light program. The third-party delivery system 52 can include, for example, hotels delivering therapy to guests via a hotel room TV or other source. The consumer direct delivery system 54 can include smart devices such as smart phones, tablets or a virtual reality headset. Each delivery system described above can initiate a request to retrieve light and sound therapy from the database 48 upon receiving a request 56 from a user to provide therapy to a patient. Upon receiving a request 56, a content delivery device can receive/retrieve content (e.g., sound and light scapes) from the database 48 in step 58 which can be experienced by a patient in step 60.

In example phase 5, a machine learning phase 5, the systems and methods of the present disclosure can monitor, in real time, a patient's feedback to a light and sound scape therapy and can adjust, also in real time, the light and sound scape therapy to the patient's vital signs 62, feedback, etc., to provide an enhanced experience uniquely and specifically tailored to the patient. The patient's vital signs can be monitored to provide input to the machine learning aspect of phase 5. This monitoring can occur through the use of biotelemetry or a wearable smart device such as a Fitibit or Apple Watch. Other devices can be used for monitoring such as a device that is placed over a person's head to monitor the person's brainwaves. Moreover, a patient can be given an input tool such as a smart phone, dial or other hardware to receive a patient's feedback 64 (e.g, a rating on a scale of 1 to 10) as to how a patient feels about the current therapy. The systems and methods can use this information as real time feedback to optimize components of sound and light for greater efficiency to allow for faster processing and a better user experience and results. Feedback includes, for example, vital sign responses and data capture on usage, abandonment, and peer rankings. Iterations, user commendations and future content creation can be driven by feedback via artificial intelligence. As can be seen, a machine learning component 66 is an artificial intelligence driven engine to optimize the sound and light scapes or files as discussed above. The machine learning component 66 can make changes to the sound library 38, light library 36, collection of associated files 40, or the database 48, among others places. In this way, as the systems and methods pull different files in real time, these files are being changed by the machine learning component 66 in real time so that the therapy being displayed to a patient is continually changing in real time based on biotelemetry, vital signs and patient feedback. Alternatively, the machine learning component 66 can choose different sound and light programs to provide the desired response to a user based on the feedback received from the user. In this way, the systems and methods of the present disclosure change files in real time on a light and/or sound scape timeline so that the light and sounds being displayed to a patient is continually changing in real time based on biotelemetry, vital signs and user feedback.

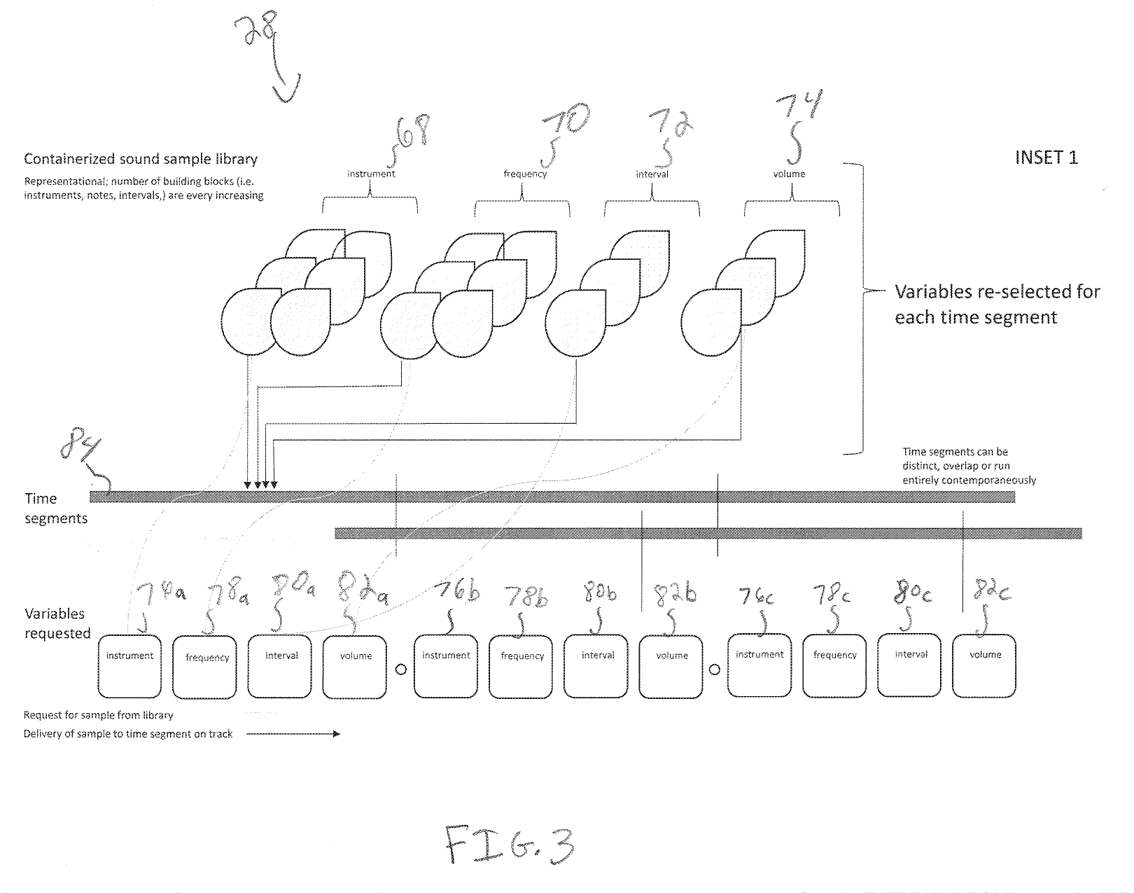

FIG. 3 is a diagram illustrating the audio library 28 used for generating the audio file 30. As can be seen in FIG. 3, the audio library 28 includes a plurality of components of sound, including, but not limited to a plurality of instruments 68, a plurality of frequencies 70, a plurality of intervals 72, and a plurality of volumes 74. This library can be added to and increased over time. Depending on the sound healing methodology 16 or the treatment path 14, the system and method can choose for a time segment a first instrument 76a of the plurality of instruments 68, a first frequency 78a of the plurality of frequencies 70, a first interval 80a of the plurality of intervals 72, and a first volume 82a, of the plurality of volumes 74. These can be selected for a first time segment 84. The same process can be repeated for subsequent time segments where different components of sound can be selected for those intervals (e.g., a subsequent time segment can have second instrument 76b, second frequency 78b, second interval 80b, and second volume 82b, and a next subsequent time segment can have third instrument 76c, third frequency 78c, third interval 80c, and third volume 82c). All of these time intervals can be combined into the audio file 30. As noted above, the systems and methods can augment the process of creating sound programs based on an intended outcome (e.g., the machine learning component 66, or some other outcome intended by the system or method of the present disclosure).

FIG. 4 is a diagram illustrating a light augmentation process 36 for generating the light file 38. The light augmentation process 36 can be based on an intended outcome (e.g., the machine learning component 66, or some other outcome intended by the system or method of the present disclosure). The light augmentation process 36 can be mapped to soundscape frequencies or a plurality of augmentation segments 84. Each augmentation segment can include a plurality of light components 86 which include but is not limited to, color, brightness, saturation, shape, duration, and movement. The plurality of augmentation segments 84 can be combined to form a light scape 88 which can form the light file 38. The light scape 88 can correspond to a sound scape 90 or audio file 30.

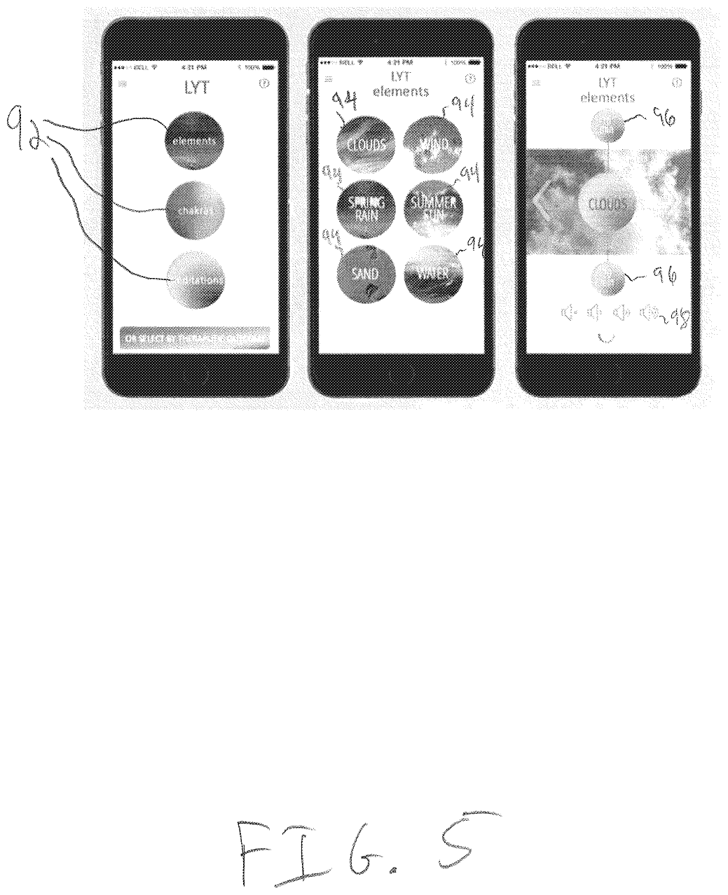

FIG. 5 is an example graphical user interface for selecting details of a sound and light therapy provided by the systems and methods of the present disclosure. In the situation of FIG. 5, a user has already selected that the condition to be treated is mental stress. Upon receiving this input, the system and method can present a plurality of themes 92 relating to this condition to be treated. In this example, the plurality of themes 92 are elements, chakras, and meditations. If the user selects elements, for example, a plurality of elements 94 can be displayed for selection by the user. An example of elements, can include, but its not limited to, clouds, wind, spring rain, summer sun, sand, and/or water. If the user selects clouds, then the system and method can present a screen which allows a user to select a length 96 of the scape to be presented to a patient. A volume control 98 can also be presented to the user.

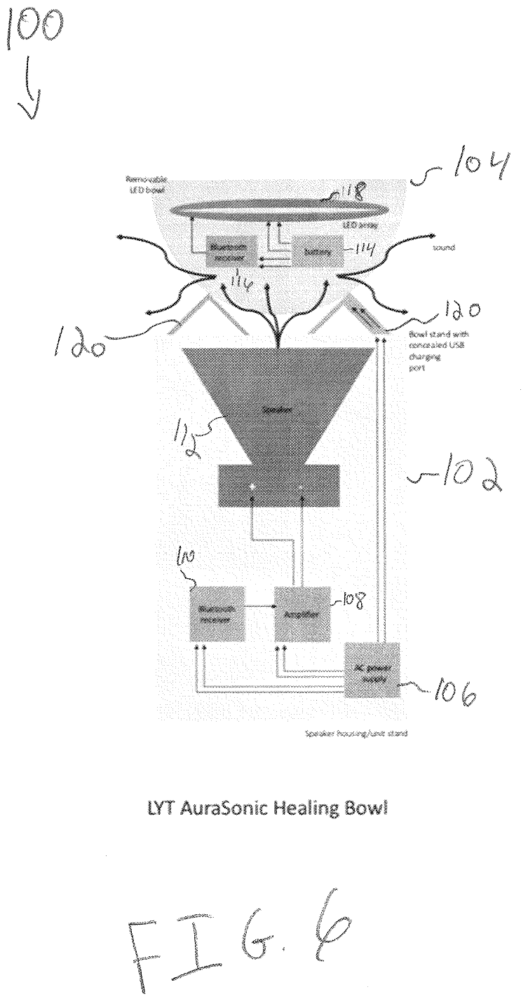

FIG. 6 is a drawing illustrating an example healing bowl 100 for providing light and sound therapy to a patient. The healing bowl 100 is an example of a therapy delivery device, but any device can be used with the systems and methods of the present disclosure as noted above (e.g., a wall). The healing bowl 100 can be a remotely controlled device to transmit chromatherapy, sound-healing and voice recording in order to elicit certain biophysical outcomes in the user. The healing bowl 100 includes a unit housing 102 and a removable LED bowl 104. The unit housing 102 includes a AC power supply 106 which can power an amplifier 108 and a Bluetooth receiver (or any other type of transceiver) 110. The Bluetooth receiver 110 receives a sound scape from a remote device which transmits the signal to the amplifier 108 which is received by a speaker 112 for producing sound healing therapy. The sound generated by the speaker 112 can be projected upward relative to the unit housing 102 which can reflect the sound off of the LED bowl 104 in a 360-degree pattern to fill the room and provide an immersive sound experience. The LED bowl 104 includes a battery 114 which provides power to a Bluetooth receiver 116 (or any other type of transceiver). The Bluetooth receiver 116 receives a light scape from a remote device which transmits the signal to a LED array 118 for producing light healing therapy (e.g., color and other elements discussed above). The LED bowl 104 can rest on a stand 120 (e.g., a three-point stand). The stand can allow power to be transmitted from the AC power supply 106 of the unit housing 102 to the battery 114 of the LED bowl 104 (via a concealing port). The user is free to remove the LED bowl 104 from the unit housing 102 and carry or hold the LED bowl 104 anywhere within Bluetooth distance and still receive the chromatherapy scape. If a user removes the LED bowl 104, the vertically positioned speaker 112 will transmit its sound directly to the room, bouncing it off the ceiling. While removed, the battery 114 which was charged by the AC power supply 106 can power the LED bowl 104. The user can control the healing bowl 100 via an application on a smart device, for example, by selecting a scape (e.g., a combined and synchronized light, sound and voice track), duration, and volume, as discussed above. The smart device communicates via Bluetooth to the Bluetooth receiver 110 housed in unit housing 102 and/or the Bluetooth receiver 116 housed in the LED bowl 104. This communication between the smart device and the Bluetooth receiver 110 and the Bluetooth receiver 116 can occur simultaneously.

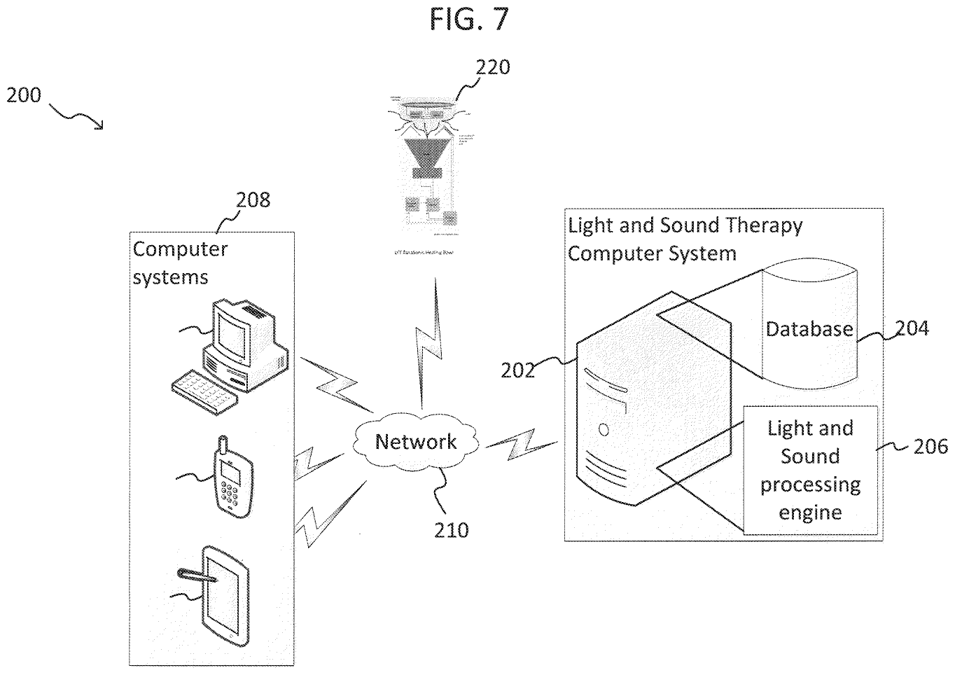

FIG. 7 is diagram illustrating hardware and software components of a system 200 of the present disclosure. The system 200 can include a light and sound therapy computer system 202. The light and sound therapy computer system can include a database 204 and a light and sound processing engine 206. The system 200 can also include a computer system(s) 208 for communicating with the light and sound therapy computer system 202 over a network 210. The computer systems 208 can be computer devices in which the participants perform the tasks as described above. Network communication could be over the Internet using standard TCP/IP communications protocols (e.g., hypertext transfer protocol (HTTP), secure HTTP (HTTPS), file transfer protocol (FTP), electronic data interchange (EDI), etc.), through a private network connection (e.g., wide-area network (WAN) connection, emails, electronic data interchange (EDI) messages, extensible markup language (XML) messages, file transfer protocol (FTP) file transfers, etc.), or any other suitable wired or wireless electronic communications format. The computer system 208 can also be a smartphone, tablet, laptop, or other similar device. The computer system 208 could be any suitable computer server (e.g., a server with an INTEL microprocessor, multiple processors, multiple processing cores) running any suitable operating system (e.g., Windows by Microsoft, Linux, etc.). The system 200 can also include a light and sound delivery device 220 (e.g., the healing bowl) for delivering light and sound therapy to a patient as discussed in greater detail above.

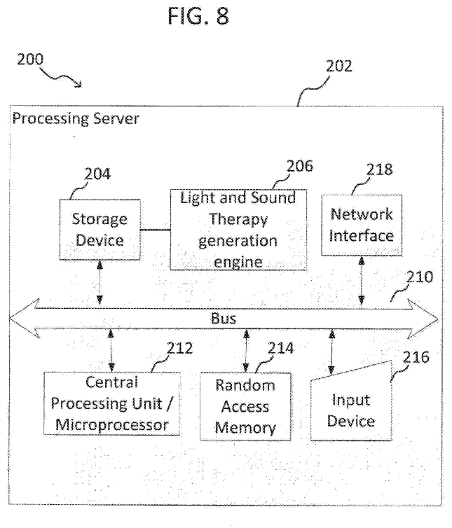

FIG. 8 is a diagram illustrating hardware and software components of a computer system on which the system of the present disclosure could be implemented. The system 200 comprises a processing server 202 which could include a storage device 204, a network interface 218, a communications bus 210, a central processing unit (CPU) (microprocessor) 212, a random access memory (RAM) 214, and one or more input devices 216, such as a keyboard, mouse, etc. The server 202 could also include a display (e.g., liquid crystal display (LCD), cathode ray tube (CRT), etc.). The storage device 204 could comprise any suitable, computer-readable storage medium such as disk, non-volatile memory (e.g., read-only memory (ROM), erasable programmable ROM (EPROM), electrically-erasable programmable ROM (EEPROM), flash memory, field-programmable gate array (FPGA), etc.). The server 202 could be a networked computer system, a personal computer, a smart phone, tablet computer etc. It is noted that the server 102 need not be a networked server, and indeed, could be a stand-alone computer system. The functionality provided by the present disclosure could be provided by a light and sound therapy program/engine 206, which could be embodied as computer-readable program code stored on the storage device 204 and executed by the CPU 212 using any suitable, high or low-level computing language, such as Python, Java, C, C++, C#, .NET, MATLAB, etc. The network interface 208 could include an Ethernet network interface device, a wireless network interface device, or any other suitable device which permits the server 202 to communicate via the network. The CPU 212 could include any suitable single- or multiple-core microprocessor of any suitable architecture that is capable of implementing and running the light and sound therapy engine 206 (e.g., Intel processor). The random access memory 214 could include any suitable, high-speed, random access memory typical of most modern computers, such as dynamic RAM (DRAM), etc.

Having thus described the system and method in detail, it is to be understood that the foregoing description is not intended to limit the spirit or scope thereof. It will be understood that the embodiments of the present disclosure described herein are merely exemplary and that a person skilled in the art may make any variations and modification without departing from the spirit and scope of the disclosure. All such variations and modifications, including those discussed above, are intended to be included within the scope of the disclosure.

1. A method comprising:
determining a condition of a patient to be treated and receiving prevailing cultural interests and evidence-based guidelines;
generating a sound healing methodology and a light healing methodology based on the condition of the patient, prevailing cultural interests, and evidence-based guidelines;
generating a sound scape from a library of sounds by selecting an instrument, a frequency, an interval, and a volume of the sound scape based on the sound healing methodology;
generating a light scape from one or more light characteristics of color, brightness, saturation, shape, duration, and movement;
generating a therapy program of light and sound to be provided to a patient by synching the light scape with the sound scape to generate the therapy program;
receiving a request to provide the therapy program to the patient;
providing the therapy program to a delivery device for providing light and sound therapy to the patient;
receiving feedback of the patient's vital signs or input feedback in real time; and
changing the therapy program in real time based on the patient's vital signs or input feedback.

2. The method of claim 1, wherein the changing step comprises:
stopping the therapy program, and
providing a second therapy program based on the patient's vital signs or input feedback.

3. The method of claim 1, wherein the changing step comprises changing the library of sounds.

4. A system for enhanced therapy optimization in connection with multisensory wellness in light and sound comprising:
a database storing a library of sounds; and
a processor in communication with the database, the processor:
determining a condition of a patient to be treated and receiving prevailing cultural interests and evidence-based guidelines;
generating a sound healing methodology and a light healing methodology based on the condition of the patient, prevailing cultural interests, and evidence-based guidelines;
generating a sound scape from the library of sounds by selecting an instrument, a frequency, an interval, and a volume of the sound scape based on the sound healing methodology;
generating a light scape from one or more light characteristics of color, brightness, saturation, shape, duration, and movement;
generating a therapy program of light and sound to be provided to a patient by synching the light scape with the sound scape to generate the therapy program;
receiving a request to provide the therapy program to the patient;
providing the therapy program to a delivery device for providing light and sound therapy to the patient;
receiving feedback of the patient's vital signs or input feedback in real time; and
changing the therapy program in real time based on the patient's vital signs or input feedback.

5. The system of claim 4, wherein changing the therapy program comprises:
stopping the therapy program, and
providing a second therapy program based on the patient's vital signs or input feedback.

6. The system of claim 4, wherein changing the therapy program comprises changing the library of sounds.

7. The system of claim 4, further comprising the delivery device.

8. The system of claim 7, wherein the delivery device comprises at least one light and at least one speaker

9. A system for enhanced therapy optimization in connection with multisensory wellness in light and sound comprising:
a database; and
a processor in communication with the database, the processor:
determining a condition of a patient to be treated,
generating a sound healing methodology and a light healing methodology based on the condition of the patient,
generating a sound scape from a library of sounds stored in the database,
generating a light scape,
generating a therapy program of light and sound by synching the generated sound scape with the generated light scape, the generated therapy program being provided to the patient,
receiving, in response to the provided therapy program, feedback of the patient's vital signs or input feedback in real time in, and
changing the provided therapy program in real time based on the patient's vital signs or input feedback.

10. The system of claim 9, wherein the processor
receives prevailing cultural interests and evidence-based guidelines, and
generates the sound healing methodology and the light healing methodology based on the condition of the patient, the prevailing cultural interests, and the evidence-based guidelines.

11. The system of claim 9, wherein the processor generates the sound scape from the library of sounds stored in the database by selecting an instrument, a frequency, an interval, and a volume of the sound scape based on the sound healing methodology.

12. The system of claim 9, wherein the processor generates the light scape from at least one of light characteristics of color, brightness, saturation, shape, duration, and movement.

13. The system of claim 9, wherein the processor receives a request to provide the generated therapy program to the patient and transmits the generated therapy program via a communication protocol to a delivery device for providing light and sound therapy to the patient.

14. The system of claim 13, further comprising the delivery device.

15. The system of claim 14, wherein the delivery device comprises at least one light and at least one speaker.

16. The system of claim 14, wherein the delivery device is a healing bowl, the healing bowl receiving the generated therapy program via Bluetooth.

17. The system of claim 9, wherein changing the therapy program comprises:
stopping the therapy program, and
providing a second therapy program based on the patient's vital signs or input feedback.

18. The system of claim 9, wherein changing the therapy program comprises changing the library of sounds.

19. The system of claim 9, further comprising the delivery device.

20. The system of claim 9, wherein the delivery device comprises at least one light and at least one speaker
